(19) United States
(12) Patent Application Publication
(10) Pub. No.: US 2019/0307688 A1
(43) Pub. Date: Oct. 10, 2019

(54) NOVEL STEM CELL CARRIER AND METHOD FOR PREPARING THE SAME

(71) Applicant: POSTECH ACADEMY-INDUSTRY FOUNDATION, Pohang-si (KR)

(72) Inventors: Hyung Joon CHA, Pohang-si (KR); Tae Yoon PARK, Daegu (KR); Hyo Jeong KIM, Busan (KR); Bong Hyuk CHOI, Pohang-si (KR)

(21) Appl. No.: 16/330,068

(22) PCT Filed: Sep. 1, 2017
(86) PCT No.: PCT/KR2017/009614

§ 371 Date: Mar. 1, 2019

(30) Foreign Application Priority Data
Sep. 1, 2016 (KR) 10-2016-0112613
Sep. 1, 2017 (KR) 10-2017-0111812

(51) Int. Cl. A61K 9/127; A61K 9/51; A61K 47/69; C12N 5/074

(52) U.S. Cl. CPC A61K 9/127; C12N 5/0607; A61K 47/6911; A61K 9/51

(57) ABSTRACT

The present disclosure relates to a novel stem cell carrier and a method for producing the same and provides a method for producing a stem cell carrier including a step of contacting stem cells with a coacervate formed by mixing an anionic polymer with a mussel adhesive protein or a mutant thereof. The present disclosure relates to a novel stem cell therapeutic agent platform of delivering cells in a encapsulated state by forming an adhesive cell carrier using crosslinked coacervate. The cell carrier of the present disclosure can maintain the ability to differentiate stem cells as well as biocompatibility and can survive without losing cell adhesion even under oxygen-deficient conditions. In addition, the cell carrier of the present disclosure has an excellent regenerative effect by applying such to biological tissues in which vascular regeneration is not easy, by inducing a metabolic reaction triggered by the hypoxic environment, in particular, neovascularization.

### TECHNICAL FIELD

The present disclosure relates to a novel stem cell carrier and a method of preparing the same.

### BACKGROUND ART

Treatment of intractable diseases with stem cells is an essential issue in the bioscience field in this century and is receiving attention in most fields of medicine, including the cardiovascular system, the nervous system, and blood. Particularly, stem cell therapy is applied even to degenerative diseases considered to be impossible to treat, thereby providing many positive results. From such results, the use of stem cells is estimated as advanced medical technology that can lead to significant changes in clinical practices based on drugs and surgery.

However, therapeutic methods that are performed using stem cells alone have been pointed out to have various problems as indicated in clinical results, and of these problems, the most significant problems are related to targetability and efficiency.

Specifically, study results revealed that conventional injection-type therapeutic agents cannot also be accurately delivered into a target site and that the injected cells can be scattered so that they are difficult to differentiate in a settled state.

Thus, as studies on solving the problem associated with the efficiency of stem cell delivery, studies on a method capable of delivering stem cells into a target site to be treated in a stable form have been conducted through cooperation in various research fields.

As a part of such efforts, hybrid tissue engineering products based on a combination of tissue engineering technology with stem cells are being actively studied. The term “tissue engineering products” refers to introducing a delivery vector into stem cells to take a suitable form of a substance having excellent biocompatibility, and then culturing the stem cells and applying the cultured stem cells to a site in need of therapy. Such tissue engineering products may be manufactured in various forms depending on disease sites.

In addition, in order for stem cells to be used as therapeutic agents for healing damaged tissues, it is essential that the stem cells maintain the ability to continue to differentiate as well as to deliver them to the injured tissue while minimizing the loss of the cells. Further, in chronic diseases for which the stem cell treatment is required, the infiltration of blood is small because not only the damaged tissue is necrotized but also the blood vessels around the tissue are also damaged. This is an environment where oxygen is insufficient for metabolism, so the cells are difficult to survive. Thus, the cell carrier for the stem cell treatment should be capable of easily transferring to target damaged tissue and having properties of having biocompatibility, minimizing cell loss, and maintaining survival and differentiation ability of stem cells. Further, in order for the stem cells and the cell carrier to be well compatible with the damaged tissue, the vasculature structure should be well-formed around the cell carrier. Thus, the development of such a cell carrier is delayed because the above-mentioned various conditions must be satisfied in order to develop a cell carrier for healing chronic diseases.

Meanwhile, a coacervate is one of the colloidal materials formed by mixing an anionic polymer electrolyte and a cationic polymer electrolyte under particular conditions. When a coacervate is formed, the absorbance of a solution increases, and it forms a spherical droplet to be separated from the solution. Upon coacervation, the participating electrolyte is separated from the solution and condensed to exist as a liquid phase. At this time, its physical properties are changed, including reduced surface tension and increased viscosity. Coacervation also occurs by mixing a protein with an oppositely charged polyelectrolyte (C. G. de Kruif et al., 2004, Current Opinion in Colloid and Interface Science 9, 340-349). Owing to low surface tension, coacervation is also employed for the microencapsulation of functional materials such as drugs, enzymes, cells, food additives or the like (Schmitt C. et al., 1998, Critical Review in Food Science and Nutrition 8, 689-753).

Marine mussels produce and secrete adhesive proteins that allow them to tightly attach themselves to wet solid surfaces such as underwater rocks, and thus fight tidal currents or buoyancy in the aqueous saline environment (J. H. Waite et al., 1983, Biological Review 58, 209-231; H. J. Cha et al., 2008, Biotechnology Journal 3, 631-638). Such mussel adhesive proteins are known as the most powerful natural adhesives, compared to the currently known chemical synthetic adhesives. Even though mussel adhesive proteins have an approximately two times higher tensile strength than epoxy resins, they are flexible. In addition, mussel adhesive proteins can also attach to various substances, including plastic, glass, metals, Teflon, and biological substances, and anchor to wet surfaces within a few minutes. These properties still remain unsolved in the fields of chemical adhesives. Mussel adhesive proteins can also be of particular value in medical applications such as adhesion of tissues or broken teeth because they do not attack the human cells or do not impose immunogenicity (J. Dove et al., 1986, Journal of American Dental Association 112, 879). In particular, mussel adhesive proteins can be used in cell surface adhesion technology, which is one of the very important technologies required in the fields of cell culture and tissue engineering. That is, the technology is to efficiently attach cells on the surface for the cell and tissue cultures, and thus the technology is critical in promoting delivery, encapsulation, proliferation and differentiation of specific cells.

Therefore, the development of stem cell carriers using cationic proteins, in particular, coacervates based on mussel adhesive proteins, could be a solution to effectively cure the living tissue.

### DISCLOSURE

### Technical Problem

Under such circumstances, the present inventors have made an effort to develop an efficient stem cell carrier. As a result, the present inventors have found that when a cell carrier is prepared by inserting stem cells into a coacervate formed by mixing a cationic mussel adhesive protein as a specific carrier and hyaluronic acid, which is one of the anionic polymers, and the cell carrier is transplanted into a living body, the in vivo delivery and encaptusulation of stem cells are remarkably increased, and the active substance is secreted from the stem cells to obtain a desired therapeutic effect, thereby completing the present disclosure.

### Technical Solution

An exemplary embodiment of the present disclosure provides a method of preparing a stem cell carrier, the method including contacting stem cells with a coacervate formed by mixing an anionic polymer with a mussel adhesive protein or a mutant thereof.

Further, another exemplary embodiment of the present disclosure provides a stem cell carrier prepared by the method as described above.

Further, yet another exemplary embodiment of the present disclosure provides a stem cell therapeutic agent including the stem cell carrier as described above.

Further, still another exemplary embodiment of the present disclosure provides a pharmaceutical composition for regenerating vascular tissue or treating a vascular abnormality-related disease, the composition including the stem cell carrier as described above.

Further, still yet another exemplary embodiment of the present disclosure provides a method for regenerating vascular tissue or treating a vascular abnormality-related disease, the method including administering a composition including the stem cell carrier as described above to the subject.

Hereinafter, the present disclosure is described in detail.

Unless otherwise defined, technical terms and scientific terms used herein have the same meaning as commonly understood by one of ordinary skill in the art to which this disclosure belongs.

Further, repeated descriptions of the same technical constitution and operation as those of the conventional art are omitted.

According to an aspect of the present disclosure, the present disclosure provides a method of preparing a stem cell carrier, the method including contacting stem cells with a coacervate formed by mixing an anionic polymer with a mussel adhesive protein or a mutant thereof and a stem cell carrier prepared according to the method as described above.

According to one preferred embodiment of the present disclosure, the stem cell carrier according to the present disclosure is a cell carrier for cell treatment containing stem cells, which is characterized to be a cell carrier prepared by the method in which stem cells are inserted inside the carrier consisting of the coacervate. i.e., stem cells are encapsulated in the coacervate. The cell carrier is transplanted in another part of a living body where the target site is not in direct contact; when the cell carrier is transplanted into a living body, the carrier prevents the encapsulated stem cells from moving from the transplantation site in the living body.

In the present disclosure, the mussel adhesive protein is one protein derived from mussels and preferably includes, but not limited to, all the mussel adhesive proteins disclosed in International Patent Publication Nos. WO2006/107183 and WO2005/092920.

According to one preferred embodiment of the present disclosure, the mussel adhesive protein or the mutant thereof may be a protein consisting of an amino acid sequence selected from the group consisting of the amino acid sequences represented by SEQ ID NO: 1, SEQ ID NO: 2 and SEQ ID NO: 3; or a fusion protein in which at least one amino acid sequence selected from the above group is linked, more preferably a protein consisting of an amino acid sequence selected from the group consisting of the amino acid sequences represented by SEQ ID NO: 1, SEQ ID NO: 2 and SEQ ID NO: 3, and most preferably a protein consisting of the amino acid sequence represented by SEQ ID NO: 1.

In the present disclosure, the mutant of the mussel adhesive protein preferably may contain additional sequences, or some amino acids substituted with other amino acids at the carboxyl terminal or amino terminal of the mussel adhesive protein under the condition that the adhesive property of the mussel adhesive protein is maintained. More preferably, a polypeptide consisting of 3 amino acids to 25 amino acids including RGD is linked to the carboxyl terminal or amino terminal of the mussel adhesive protein, or 1% to 100%, preferably 5% to 100% of the total number of tyrosine residues constituting the mussel adhesive protein may be substituted with 3,4-dihydroxyphenyl-L-alanine (DOPA).

The 3 amino acids to 25 amino acids including RGD may, but be not limited to, preferably include at least one selected from the group consisting of RGD(Arg Gly Asp), RGDS(Arg Gly Asp Ser), RGDC(Arg Gly Asp Cys), RGDV(Arg Gly Asp Val), RGDSPASSKP(Arg Gly Asp Ser Pro Ala Ser Ser Lys Pro), GRGDS(Gly Arg Gly Asp Ser), GRGDTP(Gly Arg Gly Asp Thr Pro), GRGDSP(Gly Arg Gly Asp Ser Pro), GRGDSPC(Gly Arg Gly Asp Ser Pro Cys) and YRGDS(Tyr Arg Gly Asp Ser).

The mutant of the mussel adhesive protein to which a polypeptide consisting of 3 amino acids to 25 amino acids including RGD is linked at the carboxyl terminal or amino terminal of the mussel adhesive protein may, but be not limited to, preferably include a polypeptide consisting of the amino acid sequence represented by SEQ ID NO: 2.

The mussel adhesive protein of the present disclosure may, but be not limited to, be inserted into a conventional vector which is designed for expressing an external gene so that it can be mass-produced using a genetic engineering method. The vector may be suitably selected depending on the type and characteristics of the host cell for preparing the protein or may be newly produced. A method of transforming the vector into a host cell and a method of producing a recombinant protein from the transformant may be easily performed using a conventional method. Methods of selecting, preparing, transforming, and expressing recombinant proteins as described above may be easily conducted by those skilled in the art, and some modifications in the conventional methods are included in the present disclosure.

In the present disclosure, the anionic polymer may be any polymeric substance capable of forming a coacervate by binding to the cationic mussel adhesive protein. The anionic polymer may preferably be a polymer having a pI (isoelectric point) lower than that of the cationic mussel adhesive protein, more preferably a polymer having a pI value of 2 to 6, and still more preferably a polymer having a pI value of 2 to 4. If the pI value is more than or less than the above-mentioned pI value, it is difficult to form coacervate, so that it is preferable to use an anionic polymer having the pI value within the range.

In the present disclosure, the anionic polymer may include, for example, at least one selected from the group consisting of hyaluronic acid, ferredoxin, polystyrene sulfonic acid, gum arabic, gelatin, albumin, carbopol, high or low methoxyl pectin, sodium carboxymethyl guar gum, xanthan gum, whey protein, faba bean legumin, carboxymethyl cellulose, alginate, carrageenan, sodium hexametaphosphate, sodium caseinate, hemoglobin, heparin and exopolysaccharide B40. The average molecular weight of the anionic polymer may, but not limited to, have one selected from the group consisting of 1 kDa to 300 kDa, more preferably 10 kDa to 100 kDa, still more preferably 17 kDa to 59 kDa, and most preferably 17 kDa, 35 kDa or 59 kDa. If the molecular weight thereof is more than or less than the molecular weight as described above, the coacervate may not be formed.

The stem cell carrier of the present disclosure may further include at least one kind of bioactive substance as long as the desired effect may be achieved. The bioactive substance may be a substance exhibiting a certain pharmacological activity when administered to a living body or applied to the skin surface, and may, but be not limited to, include at least one selected from the group consisting of a drug, an enzyme, a cell, and a food additive, and more preferably at least one selected from the group consisting of an anticancer agent, an antibiotic, an anti-inflammatory agent, a hormone, a hormone antagonist, interleukin, interferon, a growth factor, a tumor necrosis factor, endotoxin, lymphotoxin, urokinase, streptokinase, a tissue plasminogen activator, a protease inhibitor, alkylphosphocholine, a radioactive isotope labeling substance, a surfactant, a cardiovascular drug, a gastrointestinal drug, and a nervous system drug.

Further, the mussel adhesive protein and the anionic polymer of the cell carrier according to the present disclosure may be mixed at a weight ratio of 1:0.01 to 1:100 at a pH of 2.0 to 10.0.

The mixing process refers to a method of mixing the anionic polymer and the stem cell simultaneously with the mussel adhesive protein or mutant thereof or more preferably mixing the stem cell with the solution in which one of the mussel adhesive protein or mutant thereof and the anionic polymer is dissolved, and then further mixing the other of the mussel adhesive protein or mutant thereof and the anionic polymers to induce coacervate formation.

As described above, the coacervate prepared using the mussel adhesive protein, and the anionic polymer forms a film around the stem cells.

In addition, the mussel adhesive protein or mutant thereof and the anionic polymer may, but not be limited to, preferably be mixed at a concentration of 0.0001% by weight to 50% by weight in a solvent which is set at an optimum pH. Further, the stem cell may preferably be mixed at a volume ratio of 0.01% (v/v) to 20% (v/v), more preferably 0.1% (v/v) to 2% (v/v) in a solvent which is set at an optimum pH.

The kind, the optimum pH, and the optimum temperature of the solvent for preparing the stem cell carrier are the same as commonly known conditions in which the coacervate can be effectively formed.

As used herein, the term “stem cell” refers to a cell capable of differentiating into at least two cells while having the self-replicating capability, and may be classified as totipotent stem cells, pluripotent stem cells, and multipotent stem cells.

The stem cell of the present disclosure may be adequately selected without any limitation according to purposes and be derived from adult cells of all the known tissue or cells obtained from mammals including humans, preferably from humans, for example, from bone marrow, umbilical cord blood, placenta (or placental tissue cells), or adipose tissue (or adipose tissue cells).

For example, the stem cell may be obtained without any limitation from bone marrow, adipose tissue, muscular tissue, an ex vivo cultured autologous mesenchymal stem cell, an allogeneic mesenchymal stem cell, umbilical cord blood, embryonicyolk sac, placenta, umbilical cord, periosteum, skin from fetuses and adolescence, and blood. The stem cell may be derived from a fetus, a newborn, or an adult.

The stem cell of the present disclosure is not limited to the kind of stem cells as long as they can achieve the desired effects. However, the stem cell may preferably be one selected from the group consisting of an adipose stem cell (ASC), a mesenchymal stem cell (MSC), a bone marrow stem cell, a cord blood stem cell, a neural stem cell and an induced pluripotent stem cell, and most preferably, an adipose stem cell (ASC) or a mesenchymal stem cell (MSC).

In order to induce three-dimensional culture and engraftment of the transplanted stem cells and differentiation into specific cells, it is important to maintain a high cell density in a state of being encapsulated after transplantation. When cell-cell interactions and cell-substrate interactions are involved in inducing its adhesion, it is necessary to create an environment in which cells can proliferate while forming a three-dimensional cell cluster.

Accordingly, the present inventors have developed a method of maintaining a high cell density so as to allow cell-cell interactions and cell-substrate interactions to induce cell culture in the form of a three-dimensional cell cluster.

Therefore, the stem cell carrier of the present disclosure improves at least one stem cell ability selected from the group consisting of survival rate, proliferative ability, differentiation ability and angiogenesis ability of stem cells, thereby achieving the desired therapeutic and/or regenerative effect.

According to another aspect of the present disclosure, the present disclosure provides a pharmaceutical composition for regenerating vascular tissue or treating a vascular abnormality-related disease, the composition including the stem cell carrier.

As used herein, the term “cell therapeutic agent” refers to a pharmaceutical used for treating, diagnosing, or preventing diseases through a series of actions including changing biological properties of cells by proliferating or selecting living autologous, allogenic, or xenogenic cells in vitro or using other ways, in order to restore functions of cells and tissue. Particularly, the stem cell therapeutic agent may be classified as an embryonic stem cell therapeutic agent and adult stem cell therapeutic agent.

The stem cell therapeutic agent may be administered to a subject via any general administration route as long as it can reach the target tissue.

The administration route of the stem cell therapeutic agent may be administered intraperitoneally, intravenously, intramuscularly, or subcutaneously, but is not limited thereto.

The stem cell therapeutic agent may also be administered using any device which can deliver an active ingredient to a target cell. The stem cell therapeutic agent may be administered with a pharmaceutical carrier which is generally used for stem cell therapy. Examples of the carrier may include physiological saline solutions.

The stem cell therapeutic agent of the present disclosure can be applied directly or indirectly to the cell therapy of vascular abnormality-related diseases (for example, angiogenesis-related diseases).

The angiogenesis-related disease may be selected from the group consisting of diabetic ulcer; gangrene; wounds that require angiogenesis for healing; bureaucrats; high blood pressure; ischemic diseases including cerebral vascular ischemia, renal ischemia, pulmonary ischemia, focal ischemia and ischemic diseases including ischemic myocardial infarction; obstructive vascular diseases; and cardiovascular diseases.

In addition, the composition of the present disclosure is not limited thereto, but may be in the form of a pharmaceutical composition.

The composition of the present disclosure contains 0.0001% by weight to 50% by weight of the coacervate with respect to the total weight of the composition. The composition of the present disclosure may contain at least one active ingredient which exhibits the same or similar function in addition to the aforementioned active ingredient.

The composition of the present disclosure may be prepared by incorporating at least one pharmaceutically acceptable carrier for administration in addition to the above-described coacervate. The pharmaceutically acceptable carrier may be used as saline, sterilized water, Ringer's solution, buffered saline, dextrose solution, maltodextrin solution, glycerol, ethanol, liposome and a mixture of at least one component thereof. If necessary, other conventional additives such as an antioxidant, a buffer, and a bacteriostatic agent may be added. Further, it can be formulated into injection formulations such as aqueous solutions, suspensions and emulsions, pills, capsules, granules or tablets by additionally adding diluents, dispersants, surfactants, binders and lubricants. Target site-specific antibody or other ligands can be used in combination with the carrier. Further, it may be suitably formulated according to the respective diseases or ingredients, using appropriate methods in the art or as disclosed in Remington's Pharmaceutical Science (recent edition, Mack Publishing Company, Easton, Pa.).

The composition may be transferred to the living body by being administered via intravenous, intraperitoneal, intramuscular, subcutaneous, intradermal, nasal, mucosal, inhalation and oral pathways. The dosage varies depending on the subject's body weight, age, sex, health condition, diet, administration time, administration method, excretion rate, and disease severity. The daily dose is about 0.1 mg/kg to 100 mg/kg, preferably 0.5 mg/kg to 10 mg/kg, and the composition is more preferably administered once or several times a day.

According to yet another aspect of the present disclosure, the present disclosure provides a method for regenerating vascular tissue or treating a vascular abnormality-related disease, the method including administering a composition including the stem cell carrier as described above to the subject.

Since the method of the present disclosure uses the composition as described above, the repeated description is omitted in order to avoid the excessive complexity of the present specification.

### Advantageous Effects

According to the present disclosure, the present disclosure relates to a novel stem cell therapeutic agent platform of delivering cells in a encapsulated state by forming an adhesive cell carrier using a crosslinked coacervate. The cell carrier of the present disclosure can maintain the ability to differentiate stem cells as well as biocompatibility and can survive without losing cell adhesion even under oxygen-deficient conditions. In addition, the cell carrier of the present disclosure may show an excellent regenerative effect by applying such to biological tissues in which vascular regeneration is not easy, by inducing a metabolic reaction triggered by the hypoxic environment, in particular, neovascularization.

The foregoing summary is illustrative only and is not intended to be in any way limiting. In addition to the illustrative aspects, embodiments, and features described above, further aspects, embodiments, and features will become apparent by reference to the drawings and the following detailed description.

### DESCRIPTION OF DRAWINGS

FIG. 1 is a view showing the relationship between the concentration of cells that can be encapsulated in a coacervate and the encapsulation efficiency.

FIG. 2 is a view showing morphology of stem cells encapsulated in a coacervate through a microscope in which the scale bar is 50 μm.

FIG. 3 is a photograph showing the survival of stem cells encapsulated in a coacervate in various environments after one week has elapsed.

FIG. 4 is a view shown by quantifying the survival of stem cells encapsulated in the coacervate shown in FIG. 3.

FIG. 5 is a photograph showing an experiment in which stem cell survival and death were observed over time under low oxygen conditions.

FIG. 6 is a graph showing the results of FIG. 5.

FIG. 7 is a view showing the maintenance of stem cell differentiation ability.

FIG. 8 is a view showing the expression of SOX2 and OCT4, genes related to stem cell differentiation ability.

FIG. 9 is a graph comparing relative expression levels of Hypoxia inducible factor la gene in cells encapsulated in a coacervate.

FIG. 10 is a graph comparing relative expression levels of genes related to neovascularization such as VEGF and FGF2 in cells encapsulated in a coacervate.

FIG. 11 is a view showing the degree of neovascularization provided by treating experimental groups obtained by cutting the rat aorta.

FIG. 12 is a view showing the distribution of cells for 2 weeks using an animal light emission analyzer by injecting a stem cell carrier into a rat subcutaneously.

FIG. 13 is a view comparing fluorescently stained stem cells injected per unit area by segmenting tissue parts observed in FIG. 12.

FIG. 14 is a view showing immune response and angiogenesis through H & E staining by segmenting tissue parts observed in FIG. 12

FIG. 15 is a view showing the expression levels of proteins related to the stem cell differentiation ability in tissues after injection of the stem cell carrier into rat subcutis, and 2 weeks lapsed.

### MODES OF THE INVENTION

In the following detailed description, reference is made to the accompanying drawing, which forms a part hereof. The illustrative embodiments described in the detailed description, drawing, and claims are not meant to be limiting. Other embodiments may be utilized, and other changes may be made, without departing from the spirit or scope of the subject matter presented here.

Hereinafter, it will be apparent to those skilled in the art that Examples are only intended to explain the present disclosure in more detail, and the scope of the present disclosure is not limited by these Examples in accordance with the gist of the present disclosure.

### EXAMPLE 1

### Production of Recombinant Mussel Adhesive Protein

1.1 Production of Recombinant Mussel Adhesive Protein fp-151

The mussel adhesive protein fp-151 (SEQ ID NO:1) used in the present disclosure was produced by a method in which fp-1 variants consisting of 6 decapeptides were synthesized so that decapeptides constituted by 10 amino acids repeated 80 times in a mussel adhesive protein fp-1 present in nature to be successfully expressed in E. coli, and a gene (Genbank No. AAS00463 or AY521220) of Mgfp-5 was inserted between two fp-1 variants to be produced in E. coli (D. S. Hwang et. al., Biomaterials 28, 3560-3568, 2007).

Particularly, in an amino acid sequence of fp-1 (Genbank No. Q27409 or S23760), a fp-1 variant (hereinafter, referred to as 6xAKPSYPPTYK) in which peptides consisting of AKPSYPPTYK were repeated and linked 6 times was prepared. The 6xAKPSYPPTYK was combined to an N-terminal of Mgfp-5, and the 6xAKPSYPPTYK was combined to a C-terminal of Mgfp-5 to prepare fp-151 represented by SEQ ID NO: 1. The detailed preparation of the mussel adhesive protein was the same as those disclosed in International Patent Publication No. WO2006/107183 or WO2005/092920, the disclosures of which are incorporated herein in their entirety by reference.

1.2 Production of Recombinant Mussel Adhesive Protein fp-151-RGD

A sequence of GRGDSP selected from an RGD group was added to a C-terminal of the fp-151 prepared in Example 1.1 to prepare fp-151-RGD represented by SEQ ID NO: 2.

1.3 Production of Recombinant Mussel Adhesive Protein fp-131

The mussel adhesive protein fp-131 was produced by a method in which a gene of mussel adhesive protein Mgfp-3A present in nature (Genbank No. BAB16314 or AB049579) was inserted between two fp-1 variants to be successfully expressed in E. colias a method of preparing fp-151 described in Example 1.1.

Particularly, in an amino acid sequence of fp-1 (Genbank No. Q27409 or S23760), a fp-1 variant (hereinafter, referred to as 6xAKPSYPPTYK) in which peptides consisting of AKPSYPPTYK were repeated and linked 6 times was prepared. The 6xAKPSYPPTYK was combined to an N-terminal of Mgfp-3, and the 6xAKPSYPPTYK was combined to a C-terminal of Mgfp-3 to prepare fp-131 represented by SEQ ID NO: 3.

### EXAMPLE 2

### Preparation of Mussel Adhesive Protein-Based Coacervate

Coacervate is a kind of colloid produced by mixing anionic electrolytic polymer and cationic electrolytic polymer at specific ratios at specific pH conditions. Since the absorbance of the solution increases when the coacervate is formed, the absorbance is mainly measured to confirm the coacervate formation (V. Ducel et al., Colloids and Surfaces a-Physicochemical and Engineering Aspects, 232, 239-247, 2004).

The present inventors have confirmed the coacervate formation by mixing the mussel adhesive protein fp-151 prepared in Example 1-1 and hyaluronic acid as a negative-ion electrolytic polymer.

Specifically, hyaluronic acid having a molecular weight of 30 kDa was dissolved in a phosphate-buffered saline solution (Hyclone) at a concentration of 1% (w/v), and the mussel adhesive protein fp-151 dissolved in the above-described solution was mixed thereto while the ratio of the mussel adhesive protein fp-151 in the solute portion (the mussel adhesive protein and hyaluronic acid was increased by 10% (w/v). As a result, it was confirmed that coacervate was formed.

### EXAMPLE 3

### Preparation of Mussel Adhesive Protein-Based Coacervate-Stem Cell Carrier

The present inventors have added the mussel adhesive protein-based coacervate produced in Example 2 to mesenchymal stem cell (Sciencell #7501) medium with 1×10³ cells to 1×10⁵ cells of mesenchymal stem cells previously cultured, obtaining cell carriers in the form enclosed with a mussel adhesive protein-based coacervate. Specifically, a coacervate can be prepared by dissolving mussel adhesive protein and hyaluronic acid in a PBS solution (Hyclone) at the same ratio of 1% by weight at pH 7.2 to pH 7.4, thereby preparing coacervate having a volume ratio of mussel adhesive protein and hyaluronic acid to 7:3. The stem cell carrier may be prepared by a method in which the coacervate having non-condensed form was suspended with stem cells, and then a condensed form was produced. In addition, the stem cell carrier may be prepared by a method in which stem cells were suspended by mussel adhesive protein solution, then the hyaluronic acid solution was mixed in the volume ratio, and then the obtained coacervate was condensed.

After that, a coacervate in which cells were encapsulated was condensed by a density using a centrifuge for 3 minutes at 150 g to finally produce a stem cell carrier.

Accordingly, the concentration of cells which can be encapsulated in the coacervate and the encapsulation efficiency were confirmed.

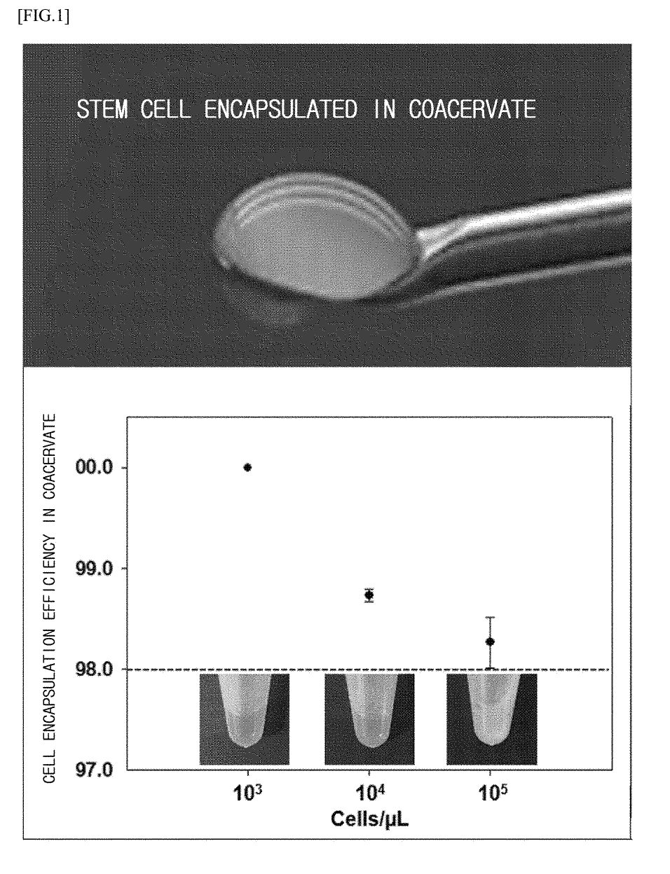

As a result, as shown in FIG. 1, since cells were easily encapsulated by low surface energy of coacervate, and then the coacervate in which cells were encapsulated could be collected, even when cells were encapsulated at 10,000 cells per unit volume (1 μL), the cell encapsulation efficiency was confirmed to be 98% or more.

Further, the morphology of the stem cells encapsulated in the coacervate was observed through a microscope.

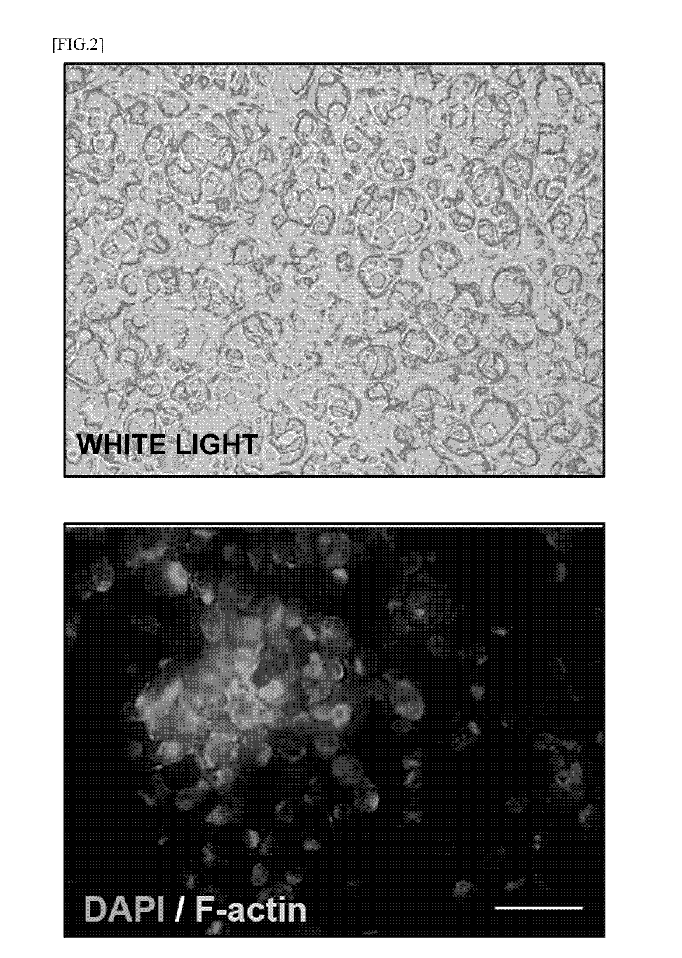

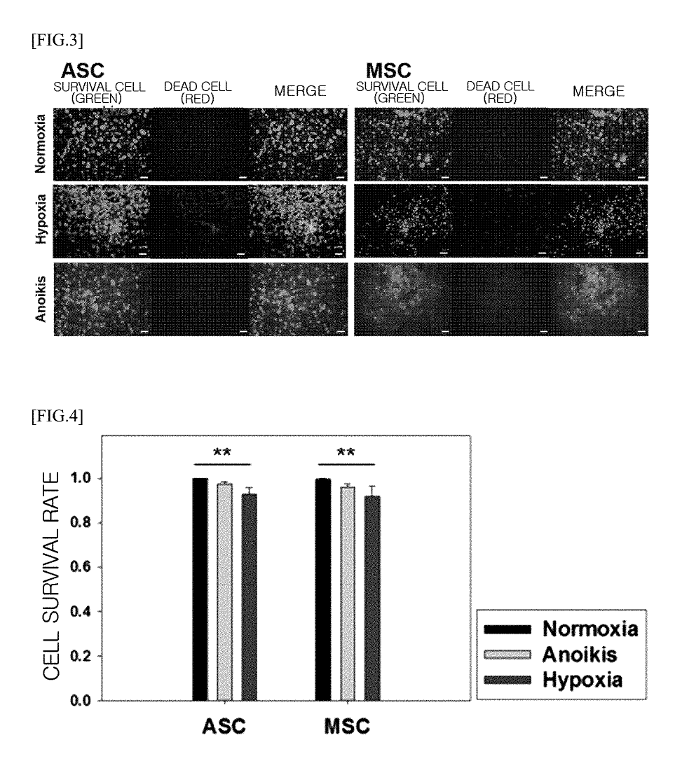

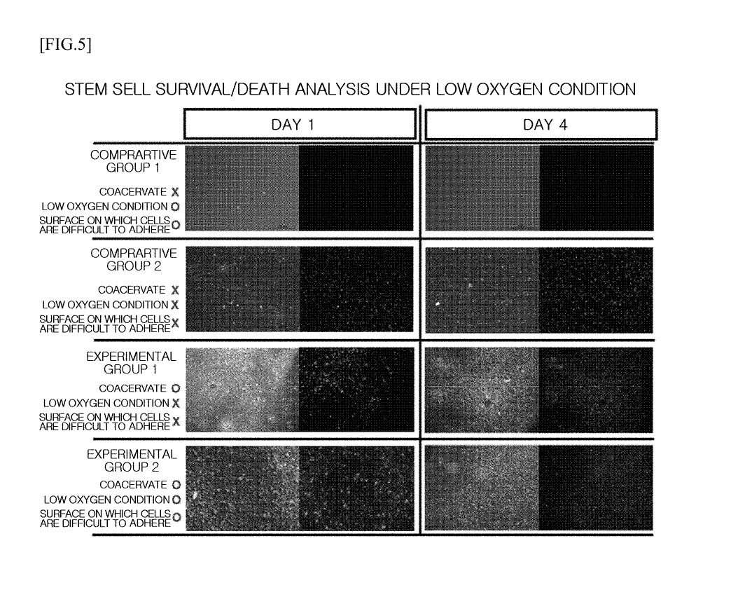

As shown in FIG. 2, the results indicated that cells were encapsulated in the water droplets in the form of a coacervate, and cell clusters were formed rather than single cells. The F-actin and nuclei of cells were stained and examined by fluorescence microscopy. As a result, it was confirmed that cell clusters were formed.

### EXAMPLE 4

### Confirmation of survival of mussel adhesive protein-based coacervate-stem cell carrier in various environments

The present inventors have observed the survival of stem cells (ASC and MSC) encapsulated in the coacervate after one week in various environments. The environment of normal oxygen level (normoxia) consists of gas 20% oxygen, 5% carbon dioxide and 75% controlled gas. The environment of low oxygen level (hypoxia) consists of 1% oxygen, 5% carbon dioxide, and 84% controlled gas. The environment of cell death (anoikis) was such that 200 μM hydrogen peroxide was added to normoxia. The surviving cells were stained with a color of green, and the dead cells were stained with a color of red. The results of whether stem cells were survival are shown in FIG. 3, and FIG. 4 is a graph showing the above results.

As a result, as shown in FIG. 3 and FIG. 4, the survival rate of stem cells encapsulated in the coacervate after 7 days was 98% in the normoxia environment, 90% in the hypoxia environment and 96% in the anoikis environment.

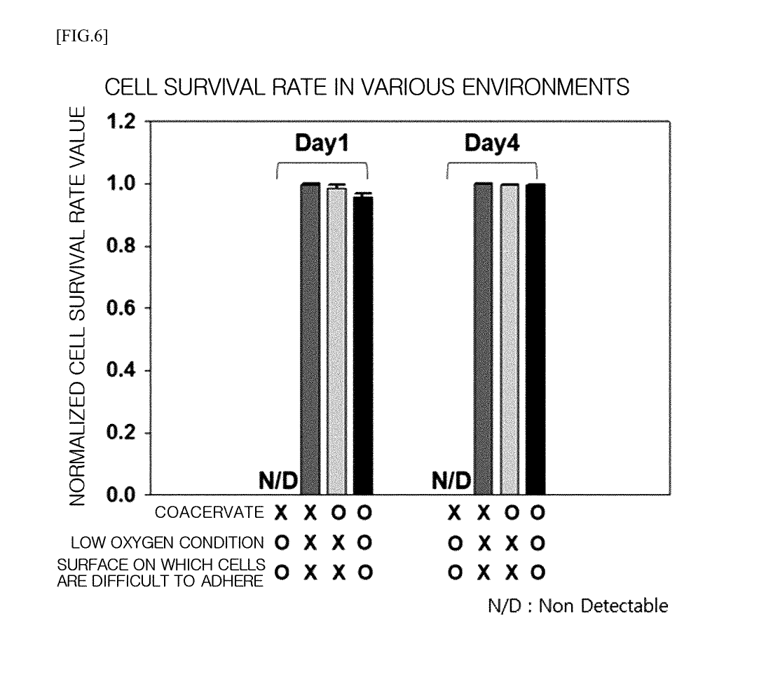

In addition, survival and death of adipose-derived mesenchymal stem cells (Handong University) and bone marrow-derived mesenchymal stem cells (Sciencell Co.) under hypoxic conditions were observed over time. The results are shown in FIG. 5, and FIG. 6 is a graph showing the results.

As shown in FIGS. 5 and 6, the cells were not observed because the cells did not adhere in Comparative group 1 in which the stem cells that were not encapsulated in the coacervate were cultured under a condition of low oxygen environment and the surface on which the cells did not adhere. The cells were found to survive well in Comparative group 2 in which the stem cells that were not encapsulated in the coacervate were cultured under a condition of sufficient oxygen environment and the surface on which the cells easily adhere. The cells were found to survive well in Experimental group 1 in which the stem cells that were encapsulated in the coacervate were cultured under a condition of sufficient oxygen environment and the surface on which the cells easily adhere. The cells were found to survive well in Experimental group 2 in which the stem cells were cultured even under a condition of low oxygen environment and the surface on which the cells were difficult to adhere.

### EXAMPLE 5

### Confirmation of Maintenance of Stem Cell Differentiation Ability of Mussel Adhesive Protein-Based Coacervate-Stem Cell Carrier

The present inventors have confirmed the stem cell function of the mussel adhesive protein-based coacervate-stem cell carrier of the present disclosure.

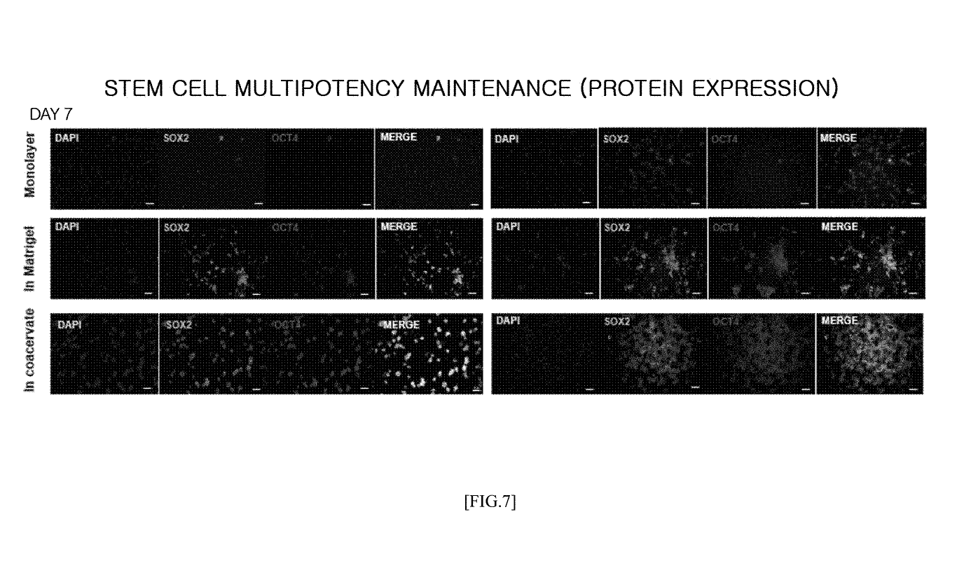

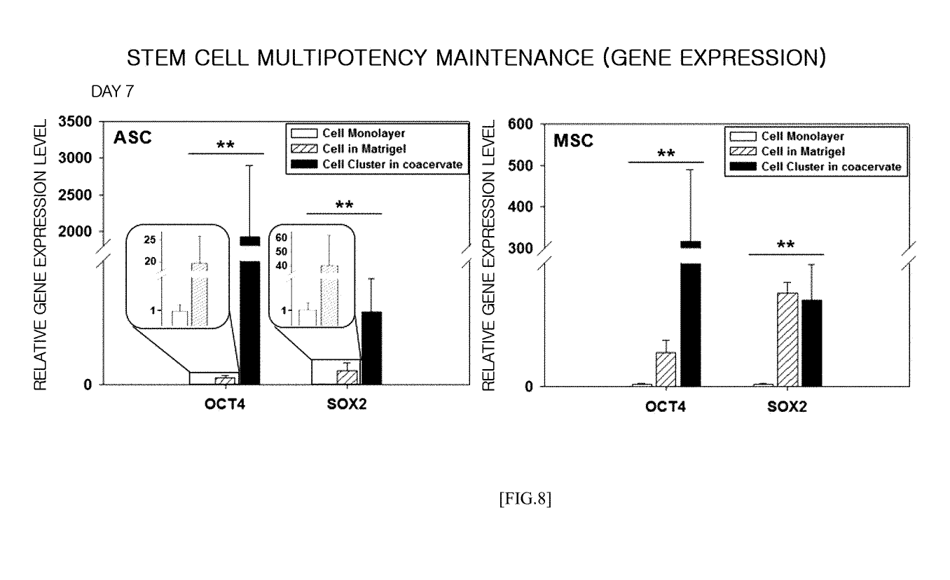

As shown in FIG. 7, the results indicated that after 7 days, the stem cells cultured under normal culture conditions, the stem cells encapsulated in Matrigel, and the stem cells injected into coacervate maintained their differentiation ability. In particular, it was confirmed that the differentiation ability of stem cells injected into coacervate was further improved. Sox2 and Oct4 were used as an antibody to confirm the maintenance of cell differentiation ability.

Further, the present inventors have confirmed the expression of SOX2 and OCT4 which are genes related to stem cell differentiation ability of the mussel adhesive protein-based coacervate-stem cell carrier of the present disclosure.

Each expression level of genes was examined with respect to stem cells cultured in the normal culture condition. As shown in FIG. 8, the results indicated that genes related to differentiation ability of stem cells encapsulated in coacervate were significantly expressed.

Further, the expression levels of Hypoxia inducible factor la gene in cells encapsulated in coacervate were relatively compared.

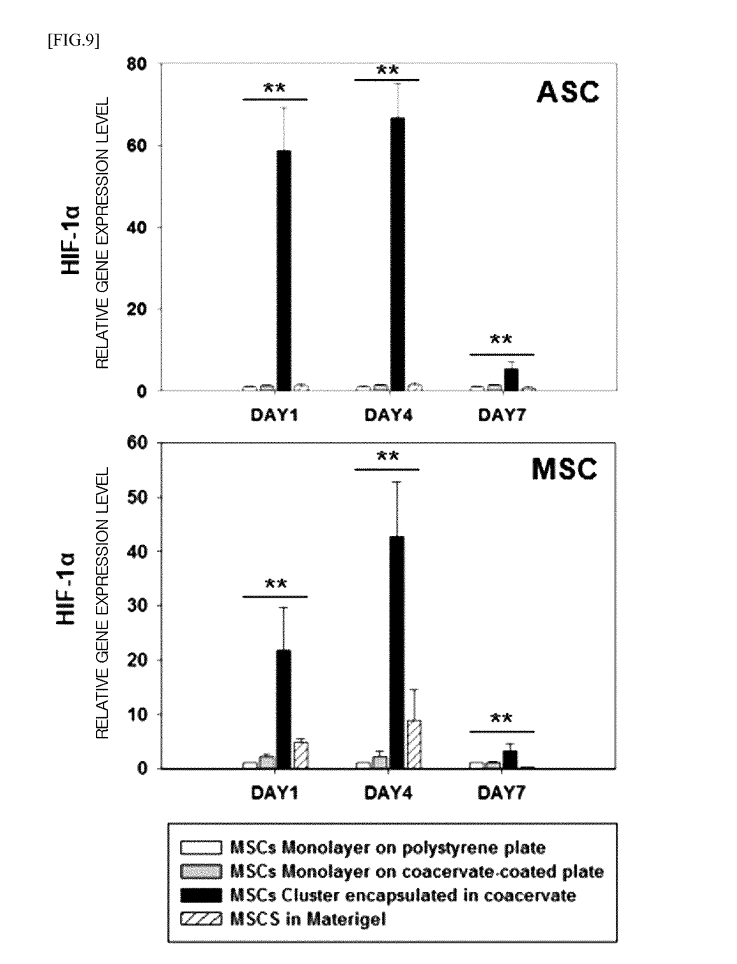

As shown in FIG. 9, the results indicated that the stem cells encapsulated in the coacervate most abundantly expressed the corresponding gene compared with the expression level in stem cells cultured on a general culture plate.

Further, the expression levels of genes involved in neovascularization such as VEGF and FGF2 were relatively compared in the cells encapsulated in the coacervate.

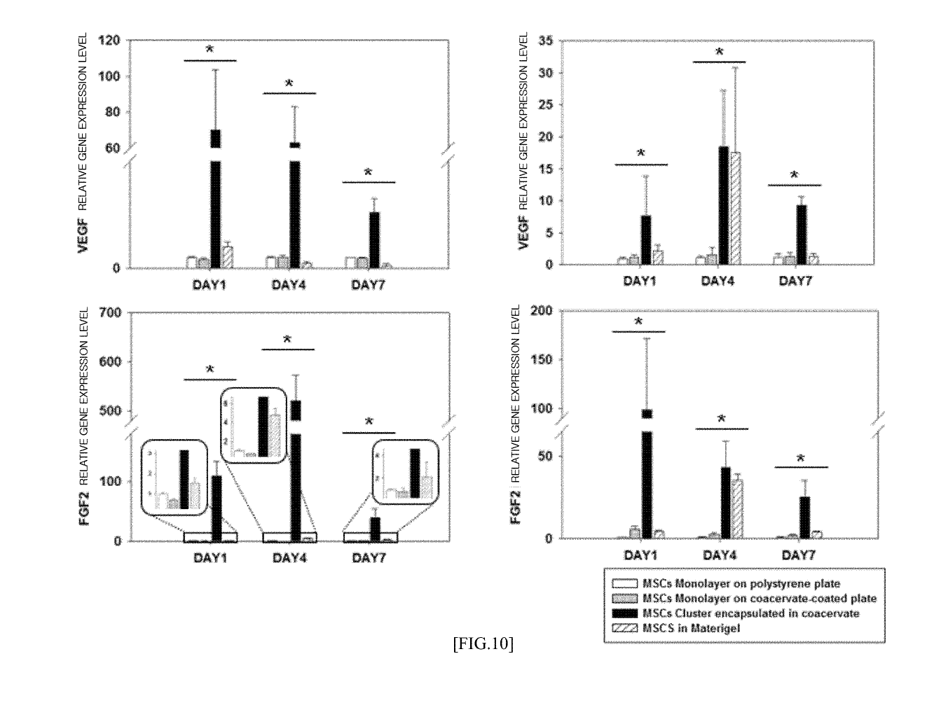

As shown in FIG. 10, the results indicated that the stem cells encapsulated in the coacervate most abundantly expressed the corresponding gene compared with the expression level in stem cells cultured on a general culture plate.

In the above experiments, primer information used for gene amplification was as follows (GAPDH was used as a housekeeping gene):

rat GAPDH (accession number: NM_017008.4)
forward primer
(SEQ ID NO. 4)
5′- GTTACCAGGGCTGCCTTCTC -3′
and
reverse primer
(SEQ ID NO. 5)
5′- GATGGTGATGGGTTTCCCGT -3′;
rat integrin β1 (accession number: NM_017022)
forward primer
(SEQ ID NO. 6)
5′- ACAAGAGTGCCGTGACAACT -3′
and
reverse primer
(SEQ ID NO. 7)
5′- CTGCAGTAAGCATCCATGTCTTCAC -3″;
rat hypoxia inducible factor-1α (HIF-1α; accession
number: NM_024359)
forward primer
(SEQ ID NO. 8)
5′- AGCAATTCTCCAAGCCCTCC -3′
and
reverse primer
(SEQ ID NO. 9)
5′- TTCATCAGTGGTGGCAGTTG -3′;
rat vascular endothelial growth factor (VEGF;
accession number: NM_001110335)
forward primer
(SEQ ID NO. 10)
5′- GCAGCATAGCAGATGTGAA -3′
and
reverse primer
(SEQ ID NO. 11)
5′- TGAACGCTCCAGGATTTA -3′;
rat fibroblast growth factor-2 (FGF-2; accession
number: NM_019305)
forward primer
(SEQ ID NO. 12)
5′- CACGTCAAACTACAGCTCCAA -3′
and
reverse primer
(SEQ ID NO. 13)
5′- GACTCCAGGCGTTCAAAGA -3′;
rat octamer-binding transcription factor-4 (OCT-4;
accession number: NM_001009178)
forward primer
(SEQ ID NO. 14)
5′- AAGTTGGCGTGGAGACTCTG -3′
and
reverse primer
(SEQ ID NO. 15)
5′- GGACTCCTCGGGACTAGGTT -3′;
rat SRY (sex determining region Y)-box 2 (SOX-2;
accession number: NM_001109181),
forward primer
(SEQ ID NO. 16)
5′- CAAGGGAATTGGGAGGGGTG -3′
and
reverse primer
(SEQ ID NO. 17)
5′- TTCATCGCCCGGAGTCTAGT -3′.

PCR was carried out on the basis of these primers, and the conditions were as follows:

PCR was performed by repeating denaturation (95° C., 10 seconds), annealing (60° C., 15 seconds), and extension (72° C., 20 seconds) for a total of 40 times to amplify genes.

Further, in order to confirm the degree of neovascularization, the rat aorta was cut and treated in each experimental group to examine whether microvessels were formed.

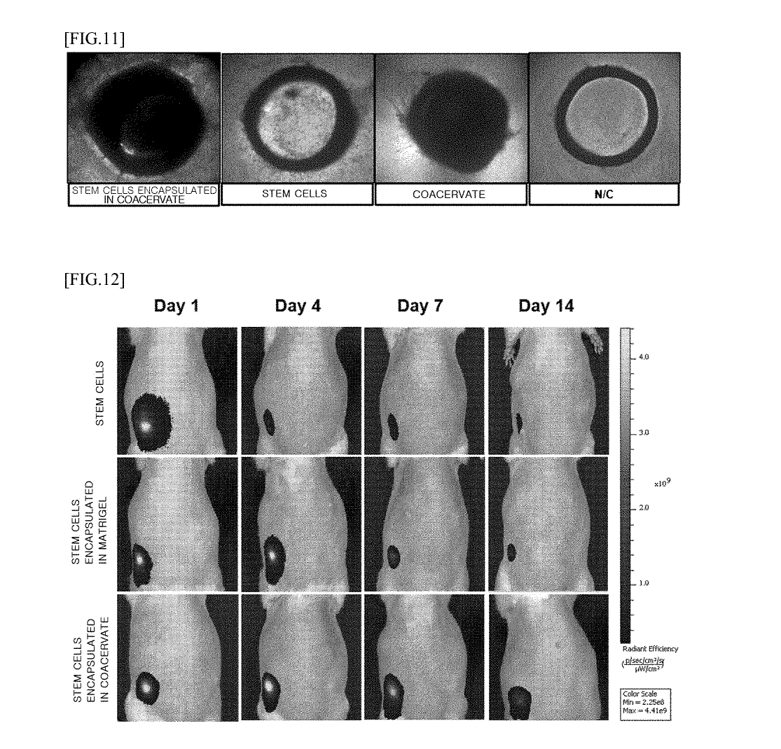

As shown in FIG. 11, the results indicated that microvessels were formed around the aorta in experimental groups treated with coacervate. In particular, it was confirmed that the microvessels were formed most abundantly in the experimental group treated with the coacervate which encapsulated the cells.

Further, stem cells were injected subcutaneously into mice, and the distribution of the cells was observed by an animal light emission image analyzer for 2 weeks. At this time, stained stem cells were used for analysis.

As shown in FIG. 12, the results indicated that even after 2 weeks had elapsed, the stem cells encapsulated in the coacervate were most distributed without scattering to the injected site.

Further, the tissue sections observed in FIG. 12 were segmented, and fluorescently stained stem cells injected per unit area were compared.

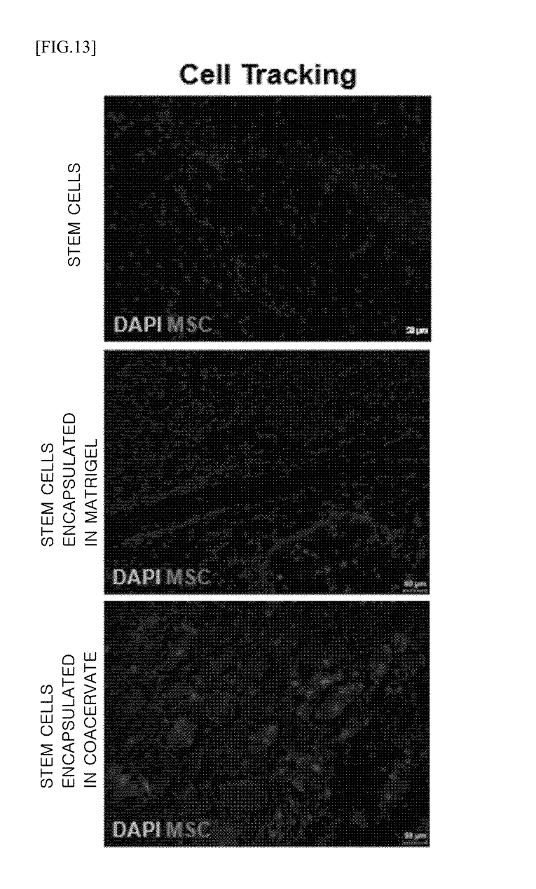

As shown in FIG. 13, the results indicated that the experimental groups in which the cells were placed together with the carrier encapsulated more cells compared with the groups in which the only cells were placed. It was confirmed that among those groups, the stem cells encapsulated in the coacervate contained the largest amount per unit area.

Further, the tissue sections observed in FIG. 12 were segmented, and the immune response and angiogenesis were examined through H & E staining.

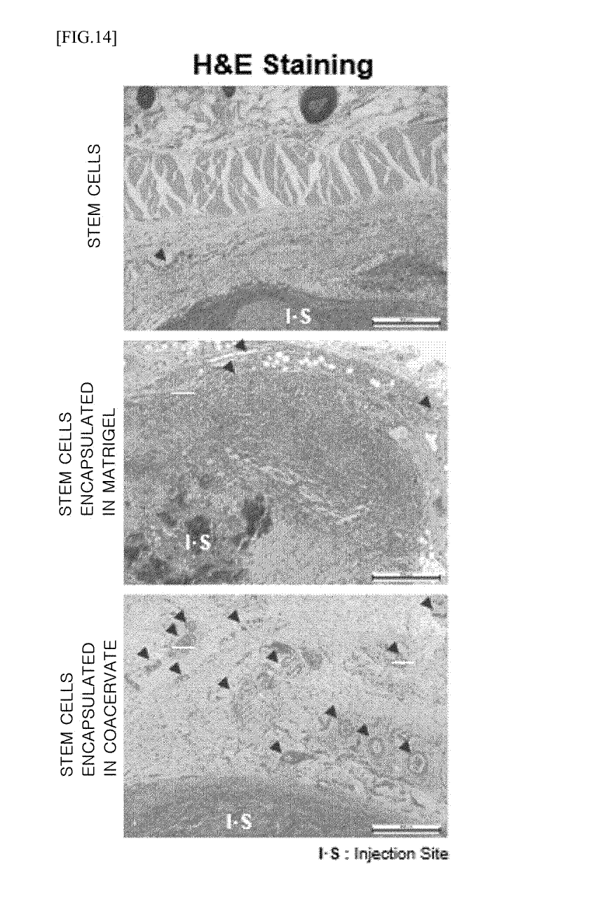

As shown in FIG. 14, the results indicated that the stem cells encapsulated in the coacervate minimized the immune response and the neovascularization was formed in the periphery thereof.

Further, stem cells were injected subcutaneously into the rat, and after 2 weeks, tissues were Western blotted to examine the protein expression levels of OCT4 and SOX2 which are related to the differentiation ability of stem cells and VEGF and FGF2 which induce neovascularization.

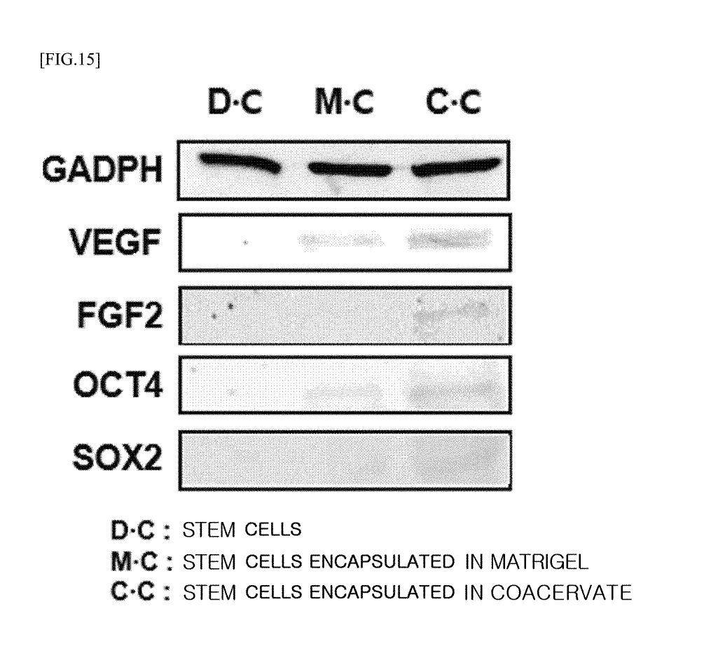

As shown in FIG. 15, the results indicated that all the proteins were most expressed in the stem cells encapsulated in the coacervate.

Therefore, the cell carrier of the present disclosure can be transplanted into a living body so that mature blood vessels can be effectively formed in the living body by an abundant angiogenesis promoting factor and vascular cells differentiated from stem cells. The cell carrier according to the present disclosure can be used not only as a cell therapeutic agent for regenerating damaged vascular tissue, but also as a composite support for tissue engineering for revascularization.

From the foregoing, it will be appreciated that various embodiments of the present disclosure have been described herein for purposes of illustration, and that various modifications may be made without departing from the scope and spirit of the present disclosure. Accordingly, the various embodiments disclosed herein are not intended to be limiting, with the true scope and spirit being indicated by the following claims.

1. A method of preparing a stem cell carrier, the method comprising:
contacting stem cells with a coacervate formed by mixing an anionic polymer with a mussel adhesive protein or a mutant thereof.

2. The method of claim 1, wherein the stem cells are encapsulated in the coacervate.

3. The method of claim 1, wherein the mussel adhesive protein or the mutant thereof is a protein consisting of an amino acid sequence selected from the group consisting of the amino acid sequences represented by SEQ ID NO: 1, SEQ ID NO: 2 and SEQ ID NO: 3; or a fusion protein in which at least one amino acid sequence selected from the group is linked.

4. The method of claim 1, wherein the anionic polymer includes at least one selected from the group consisting of hyaluronic acid, ferredoxin, polystyrene sulfonic acid, gum arabic, gelatin, albumin, carbopol, high or low methoxyl pectin, sodium carboxymethyl guar gum, xanthan gum, whey protein, faba bean legumin, carboxymethyl cellulose, alginate, carrageenan, sodium hexametaphosphate, sodium caseinate, hemoglobin, heparin and exopolysaccharide B40.

5. The method of claim 1, wherein the stem cell is one selected from the group consisting of adipose stem cell (ASC), mesenchymal stem cell (MSC), bone marrow stem cell, cord blood stem cell, neural stem cell and induced pluripotent stem cell.

6. The method of claim 1, wherein the mussel adhesive protein or the mutant thereof and the anionic polymer are mixed at a weight ratio of 1:0.01 to 1:100 at a pH 2.0 to pH 10.0.

7. A stem cell carrier prepared by the method according to claim 1.

8. A stem cell therapeutic agent including the stem cell carrier according to claim 7.

9. The stem cell therapeutic agent of claim 8, wherein the stem cell carrier improves at least one stem cell ability selected from the group consisting of survival rate, proliferative ability, differentiation ability and angiogenesis ability of stem cells.

10. A pharmaceutical composition for regenerating vascular tissue or treating a vascular abnormality-related disease, the composition comprising the stem cell carrier according to claim 8.

11. A method for regenerating vascular tissue or treating a vascular abnormality-related disease, the method comprising administering a composition including the stem cell carrier according to claim 8 to the subject.


SEQUENCE LISTING (17 sequences)

SEQ ID NO: 1 - protein, 196 aa, Artificial
  Synthetic construct - FP-151
MAKPSYPPTYKAKPSYPPTYKAKPSYPPTYKAKPSYPPTYKAKPSYPPTYKAKPSYPPTYKSSEEYKGGYYPGNTYHYHSGGSYHGSGYHGGYKGKYYGKAKKYYYKYKNSGKYKYLKKARKYHRKGYKKYYGGSSAKPSYPPTYKAKPSYPPTYKAKPSYPPTYKAKPSYPPTYKAKPSYPPTYKAKPSYPPTYK

SEQ ID NO: 2 - protein, 202 aa, Artificial
  Synthetic construct - FP-151-RGD
MAKPSYPPTYKAKPSYPPTYKAKPSYPPTYKAKPSYPPTYKAKPSYPPTYKAKPSYPPTYKSSEEYKGGYYPGNTYHYHSGGSYHGSGYHGGYKGKYYGKAKKYYYKYKNSGKYKYLKKARKYHRKGYKKYYGGSSAKPSYPPTYKAKPSYPPTYKAKPSYPPTYKAKPSYPPTYKAKPSYPPTYKAKPSYPPTYKGRGDSP

SEQ ID NO: 3 - protein, 172 aa, Artificial
  Synthetic construct - FP-131
MAKPSYPPTYKAKPSYPPTYKAKPSYPPTYKAKPSYPPTYKAKPSYPPTYKAKPSYPPTYKPWADYYGPKYGPPRRYGGGNYNRYGRRYGGYKGWNNGWKRGRWGRKYYGSAKPSYPPTYKAKPSYPPTYKAKPSYPPTYKAKPSYPPTYKAKPSYPPTYKAKPSYPPTYKL

SEQ ID NO: 4 - DNA, 20 bp, Artificial
  Synthetic construct - rat GAPDH primer F
gttaccagggctgccttctc

SEQ ID NO: 5 - DNA, 20 bp, Artificial
  Synthetic construct - rat GAPDH primer R
gatggtgatgggtttcccgt

SEQ ID NO: 6 - DNA, 20 bp, Artificial
  Synthetic construct - rat integrin b1 primer F
acaagagtgccgtgacaact

SEQ ID NO: 7 - DNA, 25 bp, Artificial
  Synthetic construct - rat integrin b1 primer R
ctgcagtaagcatccatgtcttcac

SEQ ID NO: 8 - DNA, 20 bp, Artificial
  Synthetic construct - rat hypoxia inducible factor-1a primer F
agcaattctccaagccctcc

SEQ ID NO: 9 - DNA, 20 bp, Artificial
  Synthetic construct - rat hypoxia inducible factor-1a primer R
ttcatcagtggtggcagttg

SEQ ID NO: 10 - DNA, 19 bp, Artificial
  Synthetic construct - rat vascular endothelial growth factor primer F
gcagcatagcagatgtgaa

SEQ ID NO: 11 - DNA, 18 bp, Artificial
  Synthetic construct - rat vascular endothelial growth factor primer R
tgaacgctccaggattta

SEQ ID NO: 12 - DNA, 21 bp, Artificial
  Synthetic construct - rat fibroblast growth factor-2 primer F
cacgtcaaactacagctccaa

SEQ ID NO: 13 - DNA, 19 bp, Artificial
  Synthetic construct - rat fibroblast growth factor-2 primer R
gactccaggcgttcaaaga

SEQ ID NO: 14 - DNA, 20 bp, Artificial
  Synthetic construct - rat octamer-binding transcription factor-4 primer F
aagttggcgtggagactctg

SEQ ID NO: 15 - DNA, 20 bp, Artificial
  Synthetic construct - rat octamer-binding transcription factor-4 primer R
ggactcctcgggactaggtt

SEQ ID NO: 16 - DNA, 20 bp, Artificial
  Synthetic construct - SOX-2 primer F
caagggaattgggaggggtg

SEQ ID NO: 17 - DNA, 20 bp, Artificial Sequence
  Synthetic construct - SOX-2 primer R
ttcatcgcccggagtctagt